(19) United States
(12) Patent Application Publication
(10) Pub. No.: US 2013/0172888 A1
(43) Pub. Date: Jul. 4, 2013

(54) External fixator apparatus, especially for the treatment of hand lesions.

(71) Applicant: Raul Necuze, (US)

(72) Inventor: Raul Necuze, (US)

(21) Appl. No.: 13/341,933

(22) Filed: Dec. 31, 2011

(51) Int. Cl. A61B 17/60

(52) U.S. Cl. 606/59

(57) ABSTRACT

The invention relates to an orthopedic external fixator apparatus for the treatment of diverse traumatic injuries and orthopedic pathologies of the hand, comprised of a pair of hemi-hexagonal skeletal-planes (1, 2), of which one is cephalic (1) and one is distal (2), containing several orifices (7, 10, 11) with corresponding transverse threaded (13a, 13b, 13c) orifices through which set screws (9a, 9b, 9c) secure surgical rods (8) within the skeletal-planes and both skeletal planes are connected to each other through a threaded orifice (3) by way of a pair Allen head threaded screws (4) fitted with retaining rings (5) such to allow the separation of the distal hemi-hexagon (2) when screws (4) are rotated.

### FIELD OF THE INVENTION

This invention broadly relates to an external fixator apparatus of the half ring type for stabilizing bone fractures in orthopedic surgery.

More particularly, the invention relates to unilateral type fixators specifically used for the treatment of traumatic fractures of the phalanges, hand and wrist.

As it is well known, there is a necessity to ensure maximum stability in order to provide optimum osseous fixation.

### PRIOR ART

In this specific field, unilateral fixators have been frequently employed in the treatment of hand lesions and pediatric fractures of the limbs which cannot be treated through traditional methods such as a cast.

Such external fixators comprise a unilateral bar connected to the bone via half-pins, but these unilateral fixators only provide a single plane of fixation.

Patent applications No. FR 2551345, No. GB 2138300, No. JP 1354079, No. DD 218274, No. MX159639, No. SU 1651779, No. CU 21459, No. DE 3345276 by the same applicant all disclose the same mini-external fixator for the treatment of finger fractures, whose hexagonal collars are connected to each other by two threaded rods with nuts on their ends, which allow the collars to be separated or joined serving the purpose of stabilization, but the operation of the said nuts becomes troublesome due to the necessity to be done by hand. Moreover, the hexagonal collars require manipulation and suspension of the patient's limb during surgical placement and present an increased weight and production cost. In addition, the use of stainless steel in comparison with polyacetal increases weight and decreases patient comfort. Polyacetal is also a radiolucent material which does not affect osseous visibility on x-rays.

U.S. Pat. No. 4,628,921 discloses a unilateral external fixator apparatus for hand surgery comprised of a rotating rod and at least first and second blocks with apertures on each block through which wires are surgically implanted into the bone. The problem with a simple unilateral external fixator is that the fixated region lacks stability because of its unilateral design. The underlying technical problem of this invention is to contrive a truly multi-polar fixator, for stabilizing, compressing and distracting bone fractures and providing bone lengthening capability, with functional and structural features such to allow multiple angles of fixation in order to create a stable device.

### SUMMARY OF THE INVENTION

The principle of this invention is to provide a lightweight, cost effective, simple and stable, multi-polar external fixator apparatus for the treatment of lesions of the hand and pediatric fractures.

Based on this principle, the technical problem is solved by this invention providing a fixator as previously indicated and defined in the characterizing portions of claims 1 and following.

It will be understood that the device, while illustrated as stabilizing a fracture of a phalange, can with no mechanical modifications be used to treat multiple hand and long bone orthopedic pathologies. This is due to the fact that production will include various sizes and in some cases a complete set including all necessary sizes to accommodate fractures of different sizes.

In addition, it will be further understood that the hemi-hexagonal skeletal-planes 1 and 2 will not be limited to the shape expressed in this particular embodiment and may be a full hexagon, attachable hemi-hexagons or any other possible hexagonal derived configuration.

Further features and advantages of a fixator apparatus according to the invention will be apparent from the following description of an embodiment thereof, given by way of non-limitative example with reference to the accompanying drawings. The principles and features of this invention may be employed in varied and numerous embodiments without departing from the scope of the invention.

### BRIEF DESCRIPTION OF THE DRAWINGS

Preferred embodiments of the invention are illustrated in the accompanying drawings, in which:

FIG. 1 shows a front perspective view of a hemi-hexagonal fixator apparatus according to the invention.

FIG. 2 shows a top view of the device.

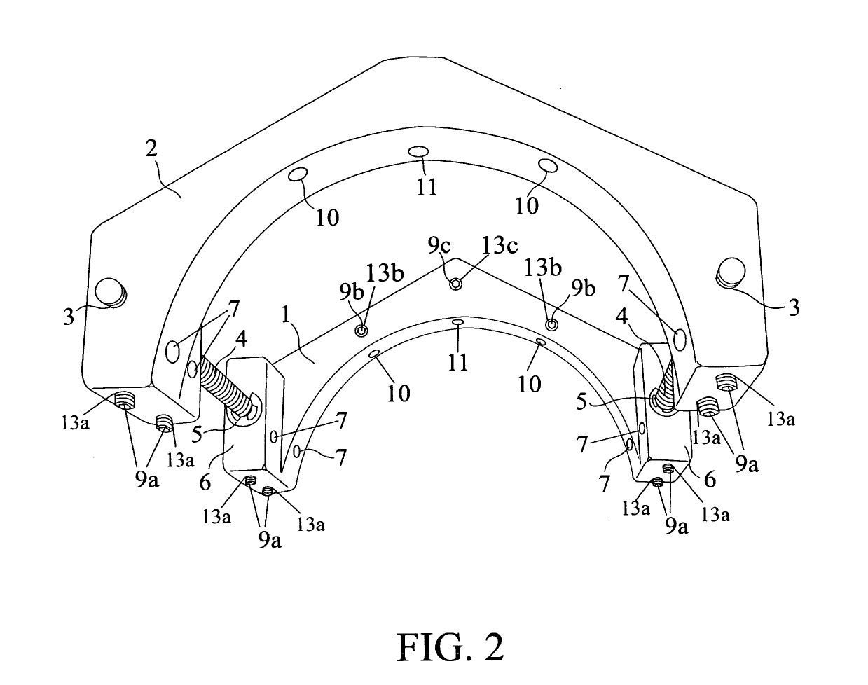

FIG. 3 shows a side view of the device.

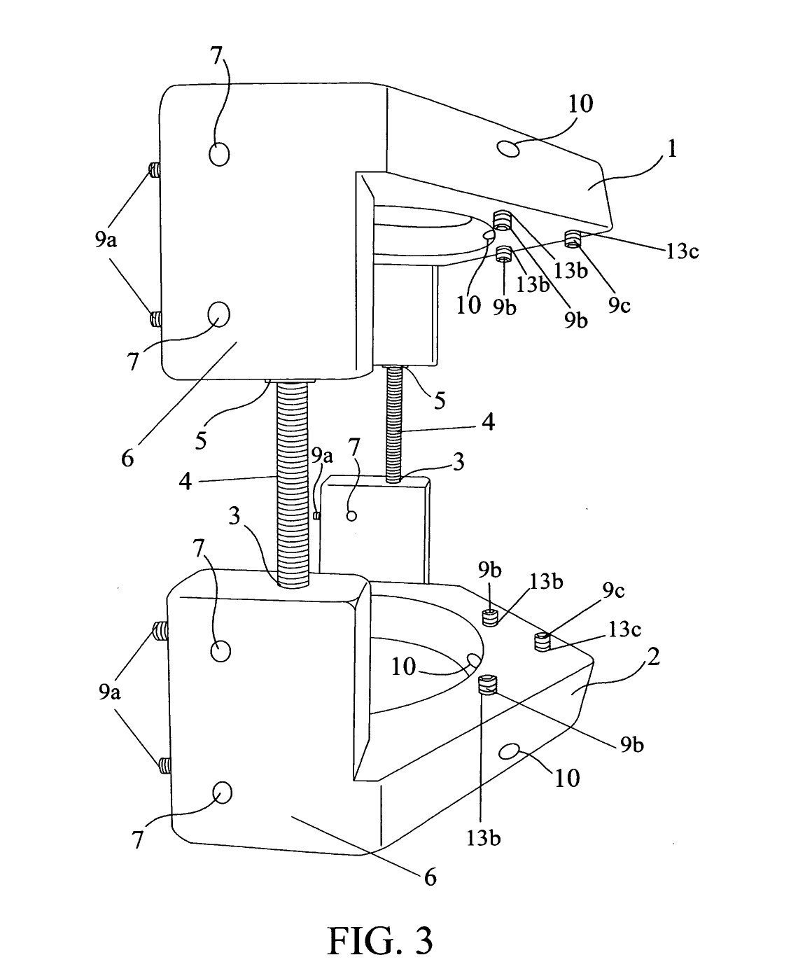

FIG. 4 shows a back view of the device.

FIG. 5 shows a top view of a hemi-hexagons skeletal-plane partially in cross section to show operation of set screws tightening surgical rods.

### DETAILED DESCRIPTION

With reference to the drawing views, a fixator apparatus according to the invention is totally shown at 1, and is of the stabilizing hemi-hexagon type.

The fixator comprises a pair of hemi-hexagonal skeletal planes 1 and 2, one cephalic 1 and one distal 2, and comprises two threaded screws 4; connecting the hemi-hexagons 1 and 2 together.

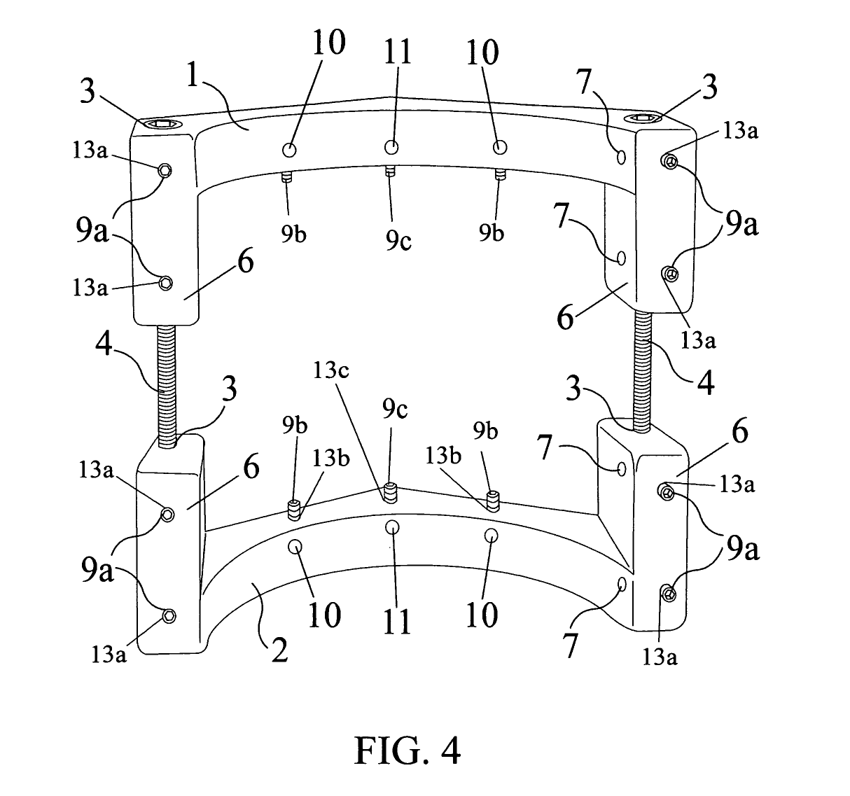

The hemi-hexagonal skeletal planes 1 and 2, have a rectangular portion 6 (see FIG. 4) vertically petruding from the ends of the legs of said parts 1 and 2. Each part 1 and 2, is therefore L-Shaped when seen from a side view.

Advantageously, both hemi-hexagons 1 and 2 are preferably manufactured out of a radiolucent material, such as polyacetal plastic with the aim to obtain clarity in osseous radiographs.

In this particular embodiment each said parts 1 and 2 are identical and half hexagonal in the example, and have multiple predetermined sizes as needed for different orthopedic pathologies.

For all descriptive purposes, it is noted that both hemi-hexagons 1 and 2 are identical in shape and that in this particular embodiment the Cephalic Hemi-Hexagon 1 is parallel to the Distal Hemi-Hexagon 2. Furthermore both frontal vertices are facing the same direction. In other words, if viewed from the top of the Cephalic hemi-hexagon one would see what would be a mirror image of said Cephalic Hemi-Hexagon 1 right below it, this would be the Distal Hemi-Hexagon 2 which is upside down in relation to Cephalic Hemi-Hexagon 1.

The said hexagons 1 and 2 have a threaded orifice 3 on both ends of their legs through which the threaded screws 4 vertically connect the said hemi-hexagons 1 and 2. The threaded screws 4 form a perpendicular angle in relation to the relative spatial position of the hemi-hexagons 1 and 2.

The threaded screws 4 are Allen screws for convenient operation with an Allen wrench.

Advantageously, screws 4 are threaded in such a way as to guarantee a predetermined distance of separation and approximation in respect to a full turn of said screws.

Of course other joining/driving combinations can be provided within the scope of the invention.

In addition, said threaded screws 4 can be made of varying lengths and widths to accommodate the corresponding needs of the varying sizes of hemi-hexagonal parts 1 and 2.

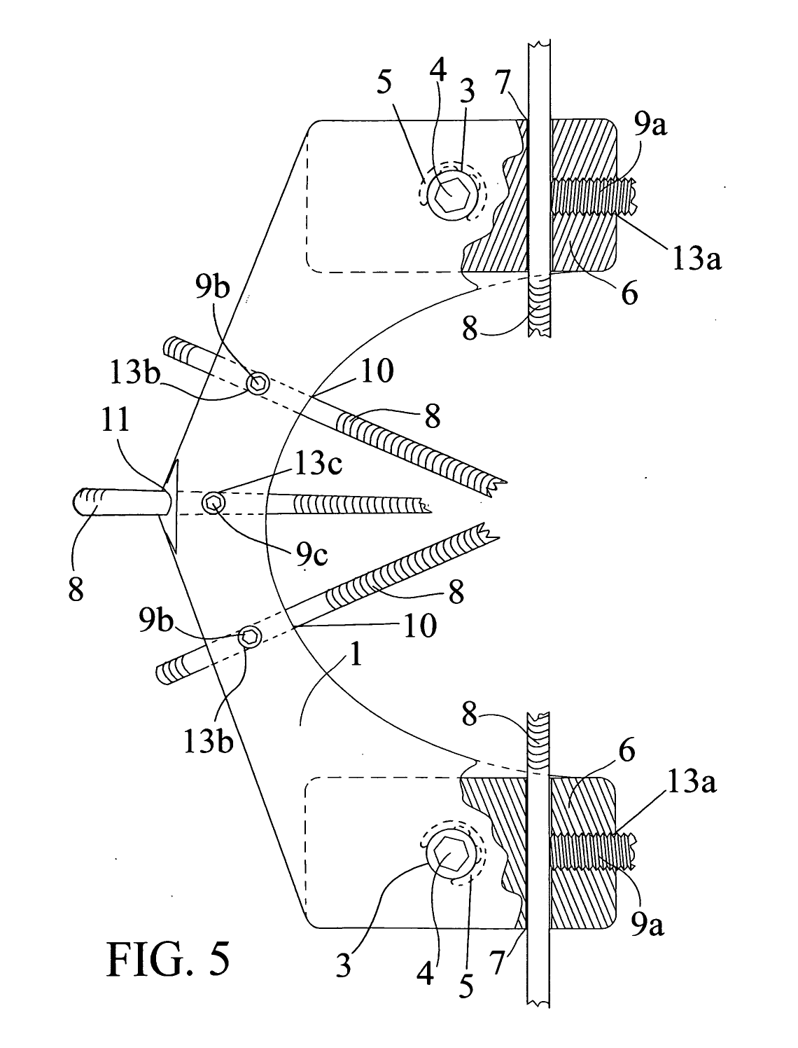

A retaining ring 5 seen in more detail in FIG. 5 is fitted on orifices 3 of the rectangular portions 6 of the cephalic hemi-hexagon 1 and around the thread of the threaded screws 4 for the purpose of restricting movement of said hemi-hexagon 1 when threaded screws 4 are rotated.

This allows separation of hemi-hexagon 2 when threaded screws 4 are rotated clockwise and approximation of hemi-hexagon 2 when screws 4 are rotated counter-clockwise, making this fixator apparatus a bone lengthening device as well.

In this particular embodiment, the said rectangular portions 6 have a pair of orifices 7 parallel to the vertical direction of screws 4 and located towards the rear section of the hemi-hexagons 1 and 2 legs and rectangular portion 6. Through said orifices 7 a surgeon inserts surgical rods 8 which completely penetrate the bone and through the use of x-ray machinery guides the said surgical rods 8 to the opposite leg of the hemi-hexagons 1 and 2, completely joining both legs of the hemi-hexagons 1 and 2 to provide the advantage of multi-polar fixation. This increases the overall stability of the device which is clearly not provided by a unilateral fixator.

For each orifice 7 there is a corresponding transverse threaded orifice 13a used to accommodate a set screw 9a (see FIG. 5) to lock a threaded surgical rod 8 to the inner walls of the orifices 7 by pressing against it from a perpendicular angle. The set screws 9a is a hex socket head for easy operation with an Allen wrench. Clockwise rotation of set screws 9a locks the surgical rods 8 in place and counter-clockwise rotation allows for loosening of surgical rods 8.

The two sides forming the vertex at the frontal face of the hemi-hexagons 1 and 2 each contain an orifice 10 in the center of said sides. Orifices 10 are horizontally oriented and angled towards the center of the hexagons 1 and 2. Furthermore, orifices 10 can be located at different levels, one being vertically positioned higher than the other to avoid collision of surgical rods.

For each orifice 10 there is a corresponding transverse threaded orifice 13b used to accommodate a set screw 9b (see FIG. 5) to lock a threaded surgical rod 8 to the inner walls of the orifices 10 by pressing against it from a perpendicular angle. The set screws 9b is a hex socket head for easy operation with an Allen wrench. Clockwise rotation of set screws 9b locks the surgical rods 8 in place and counter-clockwise rotation allows for loosening of surgical rods 8.

In this particular embodiment, the bone fixing threaded rods used in orifices 10 do not completely penetrate the bone. However they provide additional angles of fixation not available with the unilateral fixators used in current hand and finger pathologies.

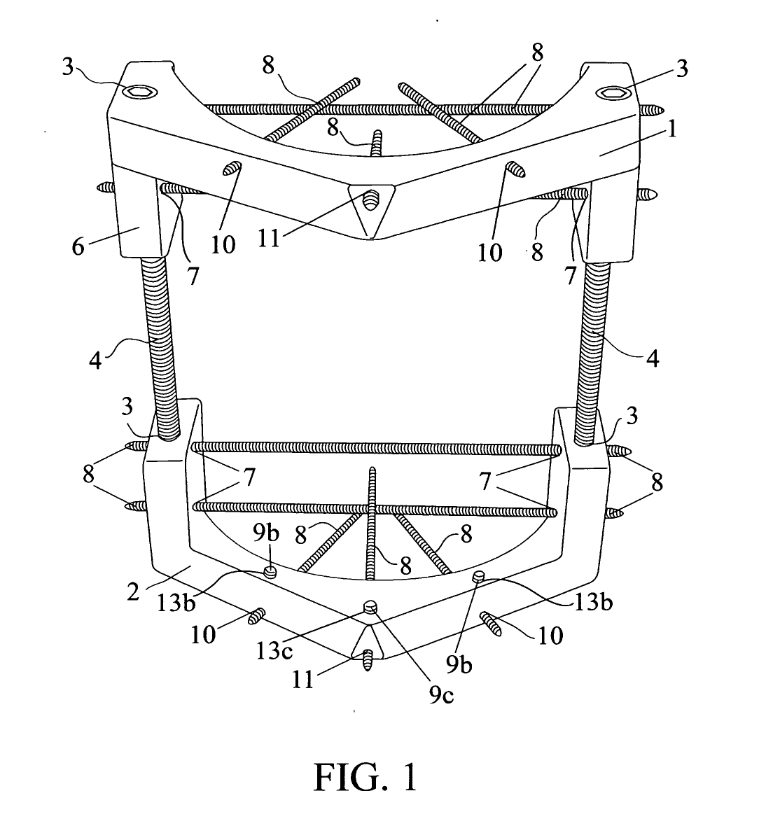

An additional orifice 11 is found on the vertex formed by the two sides of the frontal portion of the hemi-hexagons 1 and 2. The orifice 11 is angled at 15 degrees vertically towards the center of the hemi-hexagons 1 and 2 and towards the inside area of the assembled device in the particular embodiment shown on FIG. 1. This particular orifice 11 provides an additional angle through which bone fixing threaded rods can be positioned, providing even more stable osseous fixation.

For each orifice 11 there is a corresponding transverse threaded orifice 13c used to accommodate a set screw 9c (see FIG. 5) to lock a threaded surgical rod 8 to the inner walls of the orifices 10 by pressing against it from a perpendicular angle. The set screws 9c is a hex socket head for easy operation with an Allen wrench. Clockwise rotation of set screws 9c locks the surgical rods 8 in place and counter-clockwise rotation allows for loosening of surgical rods 8.

It is to be understood that the dimensions of all orifices on this invention, including but not limited to their respective diameters and angles will depend upon the location of the fracture(s) and the size of the bone to be stabilized which dictates the size of the device to be used, thereby, affecting the need to maintain proportional changes.

It is to be further understood that due to the varying amounts of pathologies treated in this particular field, the amount of orifices and location of said orifices is not to be limited to that of this particular embodiment.

It is to be further understood that the location of all set screws on this invention, will depend upon the size and use of the device which dictates the ability to use a standard size Allen wrench, thereby affecting the need to place said set screws in a different angle and location.

The external fixator apparatus illustrated in the drawings and described herein possesses numerous advantages and benefits when analyzed within the intended field of use, including:

True multi-polar fixation for treatment of traumatic and congenital hand injuries, provides multiple levels and angles of fixation.

Multi-polar and increased stability in the case of bone lengthening procedures.

Lightweight, Polyacetal material reduces weight significantly.

Radiolucent material, the use of X-ray translucent material facilitating the operative process and examination of healing progress.

Inexpensive, possibly disposable.

Ease of application, the hemi-hexagon design allows placement during the surgical process without the need to suspend or hold limb in place.

Simplicity, in this embodiment all of the functions of this device can be accomplished with only the use of Allen wrenches.

This device requires no assembly during the surgical process.

Modifications and changes to the described fixator apparatus are covered by the protection scope of the invention as defined in the claims.

The invention claimed is:

1. A multi-polar orthopedic external fixator apparatus for the treatment of diverse traumatic injuries and orthopedic pathologies of the hand, comprising: One pair of hemi-hexagonal pieces of which one is proximal and another is distal; and two threaded screws, wherein said threaded screws parallel to each other join both hemi-hexagons through threaded orifices on the legs of said hemi-hexagons, wherein retaining rings are placed on each threaded screw, wherein said hemi-hexagons have orifices through which surgical rods are inserted, wherein said surgical rods are fixed in place by set screws.

2. A multi-polar orthopedic external fixator apparatus according to claim 1, wherein said threaded screws are hex head, wherein the threading ensures that a full rotation corresponds to a predetermined distance of separation and approximation.

3. A multi-polar orthopedic external fixator apparatus according to claim 1, wherein said threaded rods contain a retaining ring to allow mobility of only one hemi-hexagon.

4. A multi-polar orthopedic external fixator apparatus according to claim 1, wherein said hemi-hexagonal pieces contain various orifices allowing for many angles and levels of osseous fixation through the use of surgical rods.

5. A multi-polar orthopedic external fixator apparatus according to claim 4, wherein said surgical rods are fixed in place by perpendicularly aligned corresponding set screws.

6. A multi-polar orthopedic external fixator apparatus according to claim 5, wherein said set screws are hex head.

7. A multi-polar orthopedic external fixator apparatus according to claims 2 and 6, wherein all components of the said hybrid external fixator may be disassembled through the use of just an Allen wrench

8. A multi-polar orthopedic external fixator apparatus according to claim 1, wherein said multi-polar orthopedic external fixator is placed fully assembled which simplifies its application in the operating room and reduces overall time of operative procedures for the patient and physician.

9. A multi-polar orthopedic external fixator apparatus according to claim 1, wherein said hemi-hexagonal skeletal planes can be constructed of a polymer X-radiation transparent material, additionally reducing weight.
